(19) United States
(12) Patent Application Publication
(10) Pub. No.: US 2009/0299252 A1
(43) Pub. Date: Dec. 3, 2009

(54) Breast Skin Care Dressing

(76) Inventor: Barrie Jane O'Neill, Boise, ID (US)

Correspondence Address: Zarian Midgley & Johnson PLLC, University Plaza, 960 Broadway Ave., Suite 250, Boise, ID, 83706, US

(21) Appl. No.: 12/408,457

(22) Filed: Mar. 20, 2009

Related U.S. Application Data
(60) Provisional application No. 61/058,325, filed on Jun. 3, 2008.

(51) Int. Cl. A61L 15/00; A61F 13/14

(52) U.S. Cl. 602/48; 602/56

(57) ABSTRACT

The present disclosure is directed to a breast skin care dressing. The breast skin care dressing comprises a moisture barrier layer having an inner surface and a perimeter. An absorbent liner covers at least a portion of the inner surface of the moisture barrier layer. The absorbent liner and the moisture barrier form a three-dimensional cup portion generally shaped for receiving a breast. An adhesive is positioned proximate the perimeter so as to be capable of maintaining a position of the absorbent liner adjacent to the breast. The present disclosure also includes methods of treating a patient using the breast skin care dressing

### BACKGROUND

The present application claims priority to U.S. Provisional Patent Application No. 61/058325, the disclosure of which is hereby incorporated by reference in its entirety.

The present disclosure relates generally to a dressing for a woman's breast.

Breast cancer patients undergoing radiation treatment can suffer from various side effects from the treatment. These side effects can include, among other things, soreness of the breast; skin irritation; dry desquamation, in which the skin of the breast can get red and raw; or moist desquamation, in which the patient may experience open sores. Treatment for these side effects can include the application of gels, creams and ointments to the breast. In the case of moist desquamation, the treatment can also include the application of dressings, such as a hydrogel absorbent pad.

These side effects can present problems for breast cancer patients. For instance, the gels, creams and ointments used for treatment can cause discomfort, are messy, and can stain or discolor clothing. Maintaining the medications and/or dressings in place on the breast can be difficult during general everyday activity. Various dressings are generally well known in the art for covering areas of the body that are treated with gels, creams and ointments. However, these dressings are typically unsuitable for use due to the shape and consistency of a woman's breast.

Dressings designed specifically for the breast are known. These dressing are often similar to a support bra. They can be designed to immobilize the breast and/or provide compression, which can be desirable for treating surgical wounds, such as those inflicted during a mastectomy. However, such dressings are often bulky and unsightly, and may not allow the user to wear certain styles of clothing. In addition, the general tenderness and sensitivity of the breast during radiation treatment can make wearing compression bandages or support garments, such as bras, uncomfortable or painful to the user.

In addition to medical dressings, other breast care products are known. For example, breast pads, which include an absorbent pad liner positioned between an outer paper moisture barrier layer and a thin membrane liner, are known for use with nursing mothers. An example of a known breast pad employs an adhesive designed to attach the breast pad to the inside cup of a bra. However, such breast pads are not necessarily suitable for treating breast cancer patients for a number of reasons. In particular, breast pads are generally made to cover the nipple area in order to prevent leakage of breast milk, and therefore are often not large enough to cover the entire breast. Further, because they are designed to attach to the cup of a bra, they are not generally suitable for use without a bra.

The present disclosure is directed to overcoming, or at least reducing the effects of, one or more of the issues set forth above.

### SUMMARY

An embodiment of the present disclosure is directed to a breast skin care dressing. The breast skin care dressing comprises a moisture barrier layer having an inner surface and a perimeter. An absorbent liner covers at least a portion of the inner surface of the moisture barrier layer. The absorbent liner and the moisture barrier form a three-dimensional cup portion generally shaped for receiving a breast. An adhesive is positioned proximate the perimeter so as to be capable of maintaining a position of the absorbent liner adjacent to the breast.

### BRIEF DESCRIPTION OF THE DRAWINGS

FIG. 1 illustrates a three dimensional view of a breast skin care dressing positioned on a patient, according to an embodiment of the present disclosure.

FIG. 2 illustrates a cross-sectional view of a breast skin care dressing, according to an embodiment of the present disclosure.

FIG. 3 illustrates a top view of a breast skin care dressing, according to an embodiment of the present disclosure.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the spirit and scope of the invention as defined by the appended claims.

### DETAILED DESCRIPTION

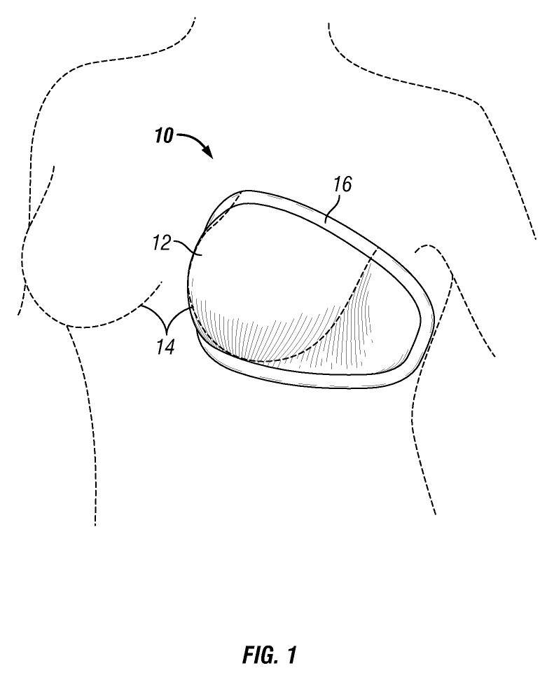

FIG. 1 illustrates a breast skin care dressing 10 according to an embodiment of the present disclosure. The breast skin care dressing can be employed by a patient to protect the breast area during treatment of any condition in which it may be beneficial to employ such a dressing. Such conditions can include skin irritation, dry desquamation and moist desquamation that can be caused by, for example, radiation treatment for breast cancer, as well as other skin conditions or minor wounds that occur in, on or around the breast.

As in the embodiment shown in FIG. 1, the breast skin care dressing 10 can include a cup portion 12 generally shaped for receiving a breast 14. A boarder portion 16 can be proximate the perimeter of the cup portion 12. The boarder portion 16 can include a means for adhering the breast skin care dressing to the patient, as will be discussed in more detail below.

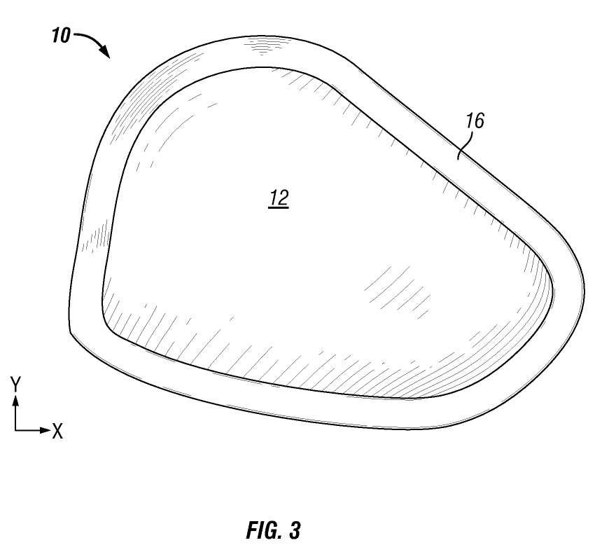

The breast skin care dressing 10 can have any suitable shape and size that can adequately cover a woman's breast. Examples of suitable shapes for covering the breast are illustrated in both FIG. 1 and FIG. 3. The shape and size can be altered to cover other regions of the body proximate the breast area, such as regions that can be affected by radiation treatment. For examples, the breast skin care dressing 10 can be shaped and sized to cover the Axilla and/or Supraclavicular regions on the patient.

Cup portion 12 is not designed to be flat, as is the case with, for example, a typical flat gauze pad. Instead, cup portion 12 comprises a three dimensional curvature to allow it to conform to breast 14. In an exemplary embodiment, when the boarder portion 16 perimeter is extended to its full length so that the dressing 10 encompasses a maximum surface area within a given x-y plane, as shown in FIG. 3, the cup portion 12 is designed to extend substantially outside of the x-y plane to form the desired cup shape.

The cup portion 12 can be manufactured in various sizes to provide a desired fit. For example, the cup portion can be designed to have a cup-size corresponding to standard bra cup sizes A, B, C or D. In an embodiment, the boarder portion 16 can wrap underneath the arm to help support and maintain the dressing in the desired position on the patient.

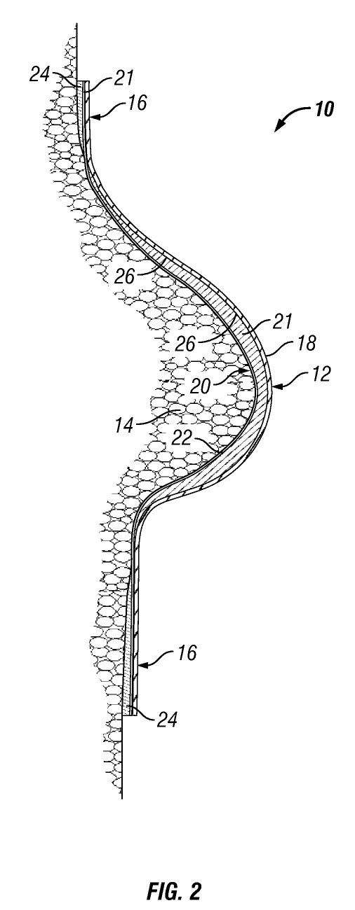

FIG. 2 illustrates a cross-sectional view of a breast skin care dressing 10, according to an embodiment of the present disclosure. As illustrated in FIG. 2, the breast skin care dressing 10 can include multiple layers, including a moisture barrier layer 18, a liner 20 and an adhesive 24.

The moisture barrier layer 18 can be any suitable layer that that can aid in providing a desired degree of protection against leakage of salves applied to the breast, thereby preventing or reducing stains and discoloring of a patient's clothing. For purposes of this application, the term “salve(s)” will refer to any suitable topical medication that may be applied to a breast for treatment of a skin condition, as well as other conditions that may occur during radiation treatment, or for treating any other medical condition of the breast, including lacerations, bruises or other wounds.

Examples of suitable moisture barrier layers can include paper, paper coated with a waxy or other polymeric material that may reduce permeation of liquids there through, or layers of polymeric material suitable for use in dressings. Examples of suitable moisture barrier layer materials are well known in the art. Moisture barrier layer 18 can comprise a single layer or multiple layers of material that together provide the desired moisture barrier characteristics. In an embodiment, moisture barrier layer 18 can provide one or more other desired properties, such as a conforming fit, breathability, stretchability, or softness.

Moisture barrier layer 18 can have any suitable size or shape that can effectively provide protection for a women's breast. In an embodiment, moisture barrier layer 18 can extend over both the cup portion 12 and border portion 16. In another embodiment, the moisture barrier layer 18 can extend only over a desired region of the breast skin care dressing, such as over the cup portion 12, but not the boarder portion 16.

Breast skin care dressing 10 can also include a liner 20, as illustrated in the embodiment of FIG. 2. In an embodiment, liner 20 can be adjacent to an inner major surface 26 of moisture barrier layer 18. Liner 20 can be of a variety of absorbencies to effectively meet the patient's needs. Liner 20 can comprise a material that can also be breathable, pliable, and/or comfortable to the patient. In an embodiment, liner 20 can be non-adhering to the skin.

The liner 20 can comprise one or more layers. In an embodiment of FIG. 2, liner 20 can including a padding layer 21 and an inner lining layer 22. Padding layer 21 can comprise an absorbing material, such as fiber padding. Examples of suitable fiber padding can include cotton padding and synthetic fiber padding, which are well known in the art. Padding layer 21 can be any suitable thickness that will provide the desired absorption capacity.

Referring to the embodiment of FIG. 2, inner lining layer 22 can comprise a membrane or thin absorbing material that can be attached to the moisture barrier layer 18. For example, inner lining layer 22 can be adhered to the moisture barrier layer 18 at the boarder portion 16 using any suitable attaching means, such as an adhesive compound or stitching (not shown). This can act to provide a pouch between the moisture barrier layer 18 and the inner lining layer 22 for holding the padding layer 21 in place. Inner lining layer 22 can comprise any suitable material, such as a fiber or mesh material that allows moisture to pass through to the padding layer 21.

In an alternative embodiment, liner 20 can comprise a single layer, such as a synthetic or cotton fiber padding, attached directly to the moisture barrier layer 18. Any suitable technique for attaching liner 20 to the moisture barrier layer can be employed. One of ordinary skill in the art would readily be able to attach liner 20 using techniques well known in the art.

In an embodiment, a thin layer (not shown) of material can be applied to liner 20 for reducing the adhesion between liner 20 and the patients skin. Such non-adhering layers are well known in the art for use in dressings generally to prevent adherence of the dressing to wounds. Any suitable non-adhering layer can be employed.

In an embodiment, the liner 20 can be pre-medicated. Packaging and selling the breast skin care dressing with a medicated liner can be convenient for the patient, as the patient can avoid having to separately apply the medication. In embodiments where multiple liner layers are employed, one or more of the liner layers can be medicated, such as, for example, inner lining layer 22. In other embodiments, the breast skin care dressing 10 can include a medicated liner layer as the sole liner layer employed.

Any suitable medication can be used, including both prescription and non-prescription, over-the-counter medications. For example, salves useful for treating skin conditions caused by radiation treatment, such as desquamination, can be employed.

The breast skin care dressing 10 can also include an adhesive 24 for adhering the breast skin care dressing 10 to a patient so as to position the liner 20 adjacent to the breast. In an embodiment, adhesive 24 can be an adhesive tape positioned at the perimeter of the cup portion 12 to form boarder portion 16. In this embodiment, the moisture barrier layer 18 may or may not extend to form part of the boarder portion 16. A well known example of a suitable adhesive tape is HYPAFIX® manufactured by Smith and Nephew Donjoy, Inc. In an embodiment, the breast skin care dressing 10 can be manufactured to include the tape positioned around the perimeter 16, and then packaged and sold to consumers.

In another embodiment, adhesive 24 can be an adhesive compound applied directly to the perimeter 16 of the breast skin care dressing. For example, adhesive 24 can be applied to the liner 20, such as inner liner layer 22, as illustrated in the embodiment of FIG. 2. Alternatively, adhesive 24 can be an adhesive compound applied directly to the portion of inner major surface 26 of moisture barrier layer 18 that forms the boarder portion 16

Any suitable adhesive can be employed, including adhesives known for use in bandages and other dressings. In an embodiment, the adhesive adheres to the skin sufficiently to hold the breast skin care dressing 10 to the patient, but provides sufficiently weak adhesion to allow removal of the breast kin care dressing 10 with a minor amount of discomfort.

A method for treating a patient having a breast condition is also disclosed. The method can include applying the breast skin care dressing 10 to a breast of a patient. In an embodiment, the patient may be undergoing radiation therapy for cancer of a breast. In an embodiment, the breast skin care dressing 10 can be pre-medicated, as discussed above. In another embodiment, a salve can be applied to the breast prior to applying the breast skin care dressing 24.

Although various embodiments have been shown and described, the present disclosure is not so limited and will be understood to include all such modifications and variations as would be apparent to one skilled in the art.

What is claimed is:

1. A breast skin care dressing, comprising:
a moisture barrier layer having an inner surface and a perimeter;
an absorbent liner covering at least a portion of the inner surface of the moisture barrier layer, the absorbent liner and the moisture barrier forming a three-dimensional cup portion generally shaped for receiving a breast; and
an adhesive positioned proximate the perimeter so as to be capable of maintaining a position of the absorbent liner adjacent to the breast.

2. The breast skin care dressing of claim 1, wherein the moisture barrier layer comprises paper.

3. The breast skin care dressing of claim 1, wherein the moisture barrier layer comprises a polymer.

4. The breast skin care dressing of claim 1, wherein the absorbent liner comprises a material chosen from cotton fibers and synthetic fibers.

5. The breast skin care dressing of claim 1, wherein the absorbent liner comprises cotton.

6. The breast skin care dressing of claim 1, wherein the adhesive is positioned around the entire perimeter of the moisture barrier layer.

7. The breast skin care dressing of claim 1, wherein the adhesive comprises an adhesive compound applied directly to the moisture barrier layer.

8. The breast skin care dressing of claim 1, wherein the adhesive is tape.

9. The breast skin care dressing of claim 1, further comprising a membrane attached to the perimeter of the moisture barrier layer, the absorbent liner being positioned between the moisture barrier layer and the membrane.

10. The breast skin care dressing of claim 1, wherein the breast skin care dressing is designed to cover only one breast of a user.

11. The breast skin care dressing of claim 1, wherein the absorbent liner is medicated.

12. The breast skin care dressing of claim 1, wherein the absorbent liner is shaped to cover at least one region of a patient in addition to the breast, the region being chosen from the axilla region and the supraclavicular region.

13. A method for treating a patient having a breast condition, the method comprising applying the breast skin care dressing of claim 1 to a breast of the patient.

14. A method for treating a patient undergoing radiation therapy for cancer of a breast, the method comprising applying the breast skin care dressing of claim 1 to the breast of the patient.

15. The method of claim 14, further comprising applying a salve to the breast prior to applying the breast skin care dressing.
